(19) United States
(12) Patent Application Publication
(10) Pub. No.: US 2024/0252782 A1
(43) Pub. Date: Aug. 1, 2024

(54) BABY SLEEP AID USING VEHICLE MOTION STIMULATOR

(71) Applicant: Salutaris Consortio Inc., Sunldand, CA (US)

(72) Inventor: Barry R. Chi, Tujunga, CA (US)

(21) Appl. No.: 18/633,491

(22) Filed: Apr. 11, 2024

Related U.S. Application Data
(63) Continuation-in-part of application No. 17/136,027, filed on Dec. 29, 2020.

(51) Int. Cl. A61M 21/02; A61M 21/00

(52) U.S. Cl. CPC A61M 21/02; A61M 2021/0022; A61M 2021/0027; A61M 2021/0044; A61M 2205/3375

(57) ABSTRACT

A sleep aid apparatus using a vehicle motion stimulator includes a sleep structure having a sleeping surface for securely confining children, a tactile transducer coupled to the sleeping structure, a vehicle simulation control unit in electrical communication with the tactile transducer, a loudspeaker and simple light projector. The vehicle simulation control unit comprises a complex audio generator configured to generate audible sonic frequency signals >100 Hz to a loudspeaker and predetermined low frequency tactile impulse from 20-100 Hz to a tactile transducer. A light projector is mounted on the unit that, when activated, produces electrical low-frequency impulses to the tactile transducer, audible signals to the ambient speaker, and electrical power to the light projector to simulate motions of bumps and rumbles with steady droning of moving car sounds and light patterns overhead simulating the experience of riding in a moving vehicle on a highway, with street lights rhythmically passing overhead.

This application claims the benefit of the filing date of currently pending provisional application No. 63/002,294, filed on Mar. 30, 2020, and is a continuation-in-part application of application Ser. No. 17/136,027.

### BACKGROUND

Motor vehicles are known to promote sleep in babies and infants when a motor vehicle is in steady motion for a period of time. This is principally due to the droning sound and vibration of the engine, various soft rumbling motions caused by the tires rolling over pavement surface irregularities and other surfaces, and in urban environments, the repeated exposure to external light sources as the vehicle passes them by. External light sources may include evenly spaced lamp lighting along freeways and highways, and/or external street lights, building lights and signage in an urban setting. It is the combined effect of light, sound, and motion that produces a calming effect in infants, babies, and small children, and promotes sleep rapidly and prolonged.

Since it is not practical or sometimes even feasible to arrange vehicle travel each time a parent or guardian desires to induce sleep for an infant, baby or small child, for napping or overnight purposes there is a need for an apparatus and method that accomplishes this task while stationary, in an interior room, such as a bedroom, nursery, or other sleeping chamber.

It is therefore an object of the present disclosure to provide an apparatus and its method of use that reliably simulates the sensory environment of a moving vehicle without the need for such vehicle travel.

The components of this vehicle motion simulation device are briefly discussed below:
1. A electromagnetic tactile transducer. It is also known as bass shakers or tactile shakers tactile transducers are designed to reproduce low-frequency vibrations in predetermined frequencies. They are typically used in applications where physical sensation is desired, such as in home theater systems, gaming chairs, and simulators. In an electromagnetic tactile transducer, an electrical current passing through a coil creates a magnetic field, which interacts with a magnet attached to a mass. This interaction causes the mass to move, generating vibrations.

Tactile transducers produce mechanical vibrations that can be fell physically by the listener and in this art, the baby. Tactile transducers are optimized for low-frequency vibrations, typically in the predetermined range of 20 Hz to 100 Hz or lower. They are often mounted directly to a surface, such as a chair or platform, to transfer the vibrations to the listener baby's body. They are a poor producer of audio sounds due to their lack of a reactive moving diaphragm such as found in a loudspeaker. It is remarkably inefficient in sound production. Its audio volume output is low even when driven at a very high signal strength. The sound frequency output is limited to very low frequencies (<100 Hz) and is muffled murky, and of unpleasant low fidelity in quality.
2. Audio loudspeakers employ an electromagnetic mechanism known as electrodynamic transduction. A loudspeaker consists of a cone-shaped diaphragm attached to a voice coil suspended within a magnetic gap. When an electrical signal is applied to the voice coil, it becomes an electromagnet and interacts with the permanent magnet, causing the voice coil (and attached diaphragm) to move back and forth rapidly. This movement of the diaphragm creates changes in air pressure, generating acoustic sound waves that propagate through the air and are perceived as sound by the listener. Audio loudspeaker covers a broader range of frequencies, typically from 20 Hz to 20,000 Hz (with most audible sound frequencies being 100 Hz.)

Loudspeakers are incapable of producing any significant tactile vibrations in response to audio signals due to their lack of a reactive magnetic mass. To create any significant, sensible tactile movements via their acoustic sound wave-induced vibrations, they must be played at an exceedingly high signal strength producing very loud sound volume (not at all conducive for sleep induction and harmful to human cars. Especially infants and baby's ears.)
3. A light source generator and projector that projects a configurable light pattern onto the ceiling or walls near the baby's carriage in a predetermined light pattern to mimic a car driving under passing street lights at night.
4. Complex Audio signal generator. This device generates a set of carefully crafted predetermined low-frequency signals ranging from 20 Hz to 100 Hz that are sent to the motion tactile transducer causing it to move and vibrate mimicking various aspects of a car ride, such as rumbling over uneven road surfaces, the coarse vibration of running over a metal grating, and the repetitive thump of driving over freeway expansion joints. The intervals of these effects are randomly generated to mimic the unpredictable nature of real-life road conditions.

This complex audio generator also generates predetermined audible signal impulses of greater than 100 Hz that are sent to the loudspeakers for them to produce a steady-state gentle sound, reminiscent of the ambient sounds riding inside of a moving vehicle.

All three effect generators are housed in the vehicle simulation control unit that is in wire connection to the tactile transducer, loudspeaker, and light projector. Each effect's random intervals and intensities are modifiable by a set of controls located on the front of the vehicle stimulation control unit based on the preferences of the baby's parents.

To achieve a realistic, and immersive car riding experience, a vehicle motion simulator must comprise a loudspeaker producing gentle ambient interior car sounds, a motion tactile transducer replicating movements akin to a car in motion, and a light projector projecting light patterns on nearby ceilings or walls to mimic driving under passing street lights at night. All three effect generators must be meticulously integrated and balance the sensory effects of sounds, movements, and lights in a cohesive and relaxing presentation that replicates the multifaceted experiences associated with being inside a vehicle in motion while also providing a calming and tranquil atmosphere conducive to comfort and sleep.

Another object of the disclosure is to provide vehicle simulated stimulation in a son-moving setting, such as a bedroom or other stationary space for sleeping. It is another object of this present disclosure to provide an apparatus that produces motion, sound and light mimicking and characteristics of typical passenger vehicle travel. These and other objects of the disclosure are more fully discussed in the following specification, claims, and drawings.

### SUMMARY

The baby sleep aid apparatus uses simulated motion vehicle (such as a car or other motion or motor vehicle) stimulation to induce and promote the rapid onset of, and continued restful sleep in infants, babies, and small children (hereinafter “children”). The apparatus includes a sleeping structure having a sleeping surface sized and configured for securely confining children. The baby sleep aid apparatus includes a tactile transducer coupled to the sleeping structure such that it vibrates the sleeping surface, thereby mimicking the interior of a motorized vehicle, when running and in motion. In various implementations, the sleeping structure may be a structure chosen from the group consisting of a baby bed, a bassinet, a cot, a cradle, and/or a crib.

The baby sleep aid apparatus also includes a vehicle simulation control unit in electrical communication with the tactile transducer. In one implementation, the vehicle simulation control unit includes a complex audio generator as discussed above, which is configured to generate predetermined tactile impulse patterns (20-100 Hz) that are then transmitted to the tactile transducer, preferably using a wired communication, which may include powering the tactile transducer in some instances. The complex audio generator also generates predetermined, soothing audio signals (100 Hz) mimicking the interior ambient sounds of a moving vehicle preferably in wired commination with loud speakers, which may or may not be attached to or within the vehicle simulating control unit. When activated, the vehicle simulation control unit simultaneously causes the tactile transducer to produce motions in the form of vibrations mimicking various movements of a car in motion as it responds to certain predetermined generated frequencies (20-100 Hz) and the loudspeaker to produce gentle ambient sounds in the audible range (>100 Hz).

The baby sleep aid apparatus also includes a simple light projector in electrical communication with the vehicle simulation control unit. In one implementation, the simple light projector is preferably mounted on the vehicle simulation control unit, and preferably on a flexible stalk configured for aiming the light in all directions. The simple light projector is configured to project a predetermined moving light pattern on nearby a ceiling or a wall when the vehicle simulator control unit is activated.

The baby sleep aid apparatus also includes a mounting apparatus configured for removably affixing the tactile transducer to a sleeping structure. In one implementation, the mounting apparatus includes a variable distance clamp bar, or other secure mounting feature, such that the produced tactile motions of the tactile transducer are transferred directly onto the sleeping structure, and thereby to the sleeping surface and infant sleeping thereon.

The tactile (i.e., vibration) transducer impulse patterns, and any desired ambient vehicle or other sounds, are generated by the complex audio generator that lies within the vehicle simulation control unit. The complex audio generator is configured to be able to modify the impulse and sound envelope patterns to the desired effect as dictated by the user. Moreover, the vehicle simulation control unit also provides an electrical signal to the simple light projector wherein the light pattern, such as simulating street lights rhythmically passed by a vehicle, may also be modified by the user.

For programmable control, the vehicle simulation control unit also preferably includes an SD card render or similar memory device, including removable memory card drive, whereby a user may be able to upload additional alternative vehicle motion simulation data into the vehicle simulation control unit so to produce motor vehicle environments with different characteristics. The vehicle simulation control unit is preferably configured with an appropriate user interface for modifying relevant sound and motion parameters. In one implementation, the control unit includes the ability to accept an alternative vehicle simulation data stream that is pre-recorded by the user (or created by a third party, including simulation data provided by the manufacturer) to provide a user with specific motor vehicle characteristics known to be effective.

In certain implementations, the vehicle simulation control unit may include a receiver that may be in wireless communication with user's mobile phone where an applicable mobile app downloaded to the user's mobile phone may remotely communicate and modify the signal output of the vehicle simulation control unit. In other implementations the vehicle simulation control unit may include a microphone, which may be external, and it is configured to detect infant crying sounds with adjustable sensitivity that once detected infant's crying sounds, automatically enables the vehicle motion simulator for a modifiable, predetermined duration as set forth by the user to sooth the infant baby back to sleep. At the end of the selected duration, the simulated car effects are gradually reduced over a certain period to minimize the jolt of au abrupt shutdown. Additionally, there is also an internal time clock where the user may set the operational duration of the simulator (minutes to hours) before automatic gradual shutdown in the manner noted above.

### BRIEF DESCRIPTION OF THE FIGURES

FIG. 1 illustrates a baby sleep aid using simulated car stimulation apparatus.

FIG. 2 illustrates a tactile transducer affixed to a mounting apparatus for mounting the apparatus onto a sleep structure.

FIG. 3 illustrates attachment sites for the mounting apparatus.

FIG. 4 illustrates alternative attachment sites for the mounting apparatus.

FIG. 5 illustrates an additional alternative attachment site for the mounting apparatus.

FIG. 6 illustrates an additional alternative attachment site for the mounting apparatus.

FIG. 7 illustrates an additional alternative attachment site for the mounting apparatus.

FIG. 8. illustrates a schematic diagram of the second implementation baby sleep aid.

### REFERENCE NUMBERS

10. baby sleep aid apparatus
12. sleeping structure
14. sleeping surface.
16. tactile transducer
18. vehicle simulation control unit
20. complex audio generator
22. loudspeaker
24. mounting apparatus
26. first cord
27. second cord
28. first clamp assembly
30. second clamp assembly
32. bracket
34. wingnut
36. wingnut track.
38. light source
40. flexible mount
46. SD card reader.
48. power supply
50. microphone

### DESCRIPTION

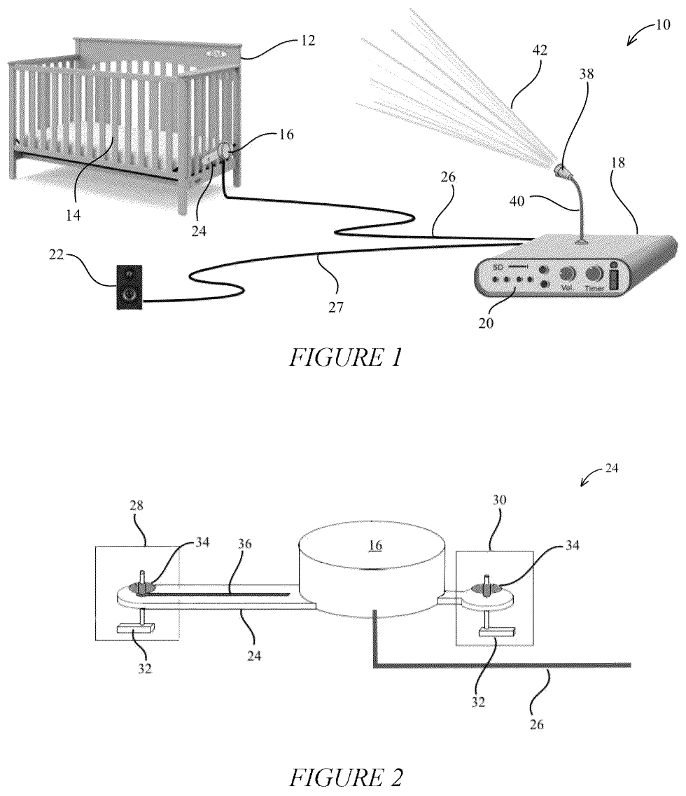

Referring to FIG. 1, a baby sleep aid apparatus 10 using a motion vehicle stimulator to promote sleep in infants is shown. The baby sleep aid apparatus 10, includes a structure 12 having a sleeping surface 14 for an infant. In one implementation, the sleeping surface 14 is preferably sized and configured as a bed as illustrated. The sleeping structure 12 is sufficiently confining to maintain the infant in the bed sleeping structure 12. A tactile transducer 16 is coupled to the structure 12. The tactile transducer 16 is securely affixed to the sleeping structure 12, such that the vibrations, and tactile impulses produced by the tactile transducer 16 are transferred to the sleeping structure 12, thereby causing the sleeping surface 14 to gently vibrate and bump. The baby sleep aid apparatus 10 includes a light source 38, which may be a lamp or similar light producing apparatus. The light source 38 may be moving light pattern 42 that simulates street lamps as a moving vehicle travels under them. The baby sleep aid apparatus 10 may include a speaker wire 27 connecting the vehicle simulation control unit 18 to the speaker 22.

Still referring to FIG. 1, in the illustrated embodiment, the tactile transducer 16 is in wired communication with a vehicle simulation control unit 18. The vehicle simulation control unit. 18 includes a complex audio generator 20, configured to generate tactile motions in predetermined low frequencies (20 Hz-100 Hz). These tactile impulse patterns (not shown) are communicated to the tactile transducer 16, thereby causing the tactile transducer 16 to vibrate and bump according to predetermined but modifiable tactile patterns. The complex audio, generator 20 has the capacity to modify the elements of the tactile wave envelope (i.e., attack, sustain, decay, release and frequency), including the intensity of the tactile wave itself.

The complex audio generator 20 within the vehicle simulation control unit. 18 includes user interface control that allows users to shape the predetermined, low-frequency wave (20 Hz-100 Hz) envelopes generated by the complex audio generator 20, producing differing tactile motions, as well as, variation in audible ambient sound waves (>100 Hz) that are transmitted to the sleeping structure 12. Furthermore, the light patterns of the simple projector may also be modified by the vehicle simulation control unit 18.

In one implementation, the vehicle simulation control unit 18 also includes wireless functionality that is capable of modifying all the effects such as sounds, motions, and light pattern's produced by the vehicle motion simulator based on the user's preferences. In other implementations, this may be accomplished remotely, by the use of user's mobile phone and appropriate mobile app.

The vehicle simulation control unit 18 is also preferably in wired communication with a speaker 22, using a second cord 27. Thus, when the vehicle simulation control unit 18 is activated, it simultaneously causes the tactile transducer 16 to produce motions and movements in response to predetermined, modifiable low frequencies (20-100 Hz) impulses, and also causes the speaker 22 to produce an accompanying audible sound frequency (>100 Hz) In one implementation, this may be an ambient internal motor vehicle sound in an audible range.

Referring to FIG. 2, the tactile transducer 16 is shown affixed to a mounting apparatus 24. The tactile transducer 16 is preferably enclosed in a magnetically shielded shell to avoid strong magnetic forces near the sleeping structure 12 and sleeping surface 14. Also shown in the illustrated view, a first cord 26 is shown connecting the tactile transducer 16 to the vehicle simulation control unit 18. The first cord 26 is preferably water proof and sufficiently resilient for repeated use.

The mounting apparatus 24, in the illustrated embodiment, comprises a substantially flat elongated member having a first clamp assembly 28 and a second clamp assembly 30 at either end. The mounting apparatus 24 with its elongated member is strong and inflexible in order to transfer all vibrations and tactile motions from the tactile transducer 16 to the sleeping structure 12 via the first clamp assembly 28 and the second clamp assembly 30. The first clamp assembly 28 and the second clamp assembly 30 each comprise a bracket 32 and a wingnut 34 for tightening the bracket against the sleeping structure 12 (not shown). A wingnut track 36 is provided for sliding the first clamp assembly 28 proximal or distal to the tactile transducer 16 in order to accommodate the mounting apparatus 24 onto differing sleep structure 12 dimensions.

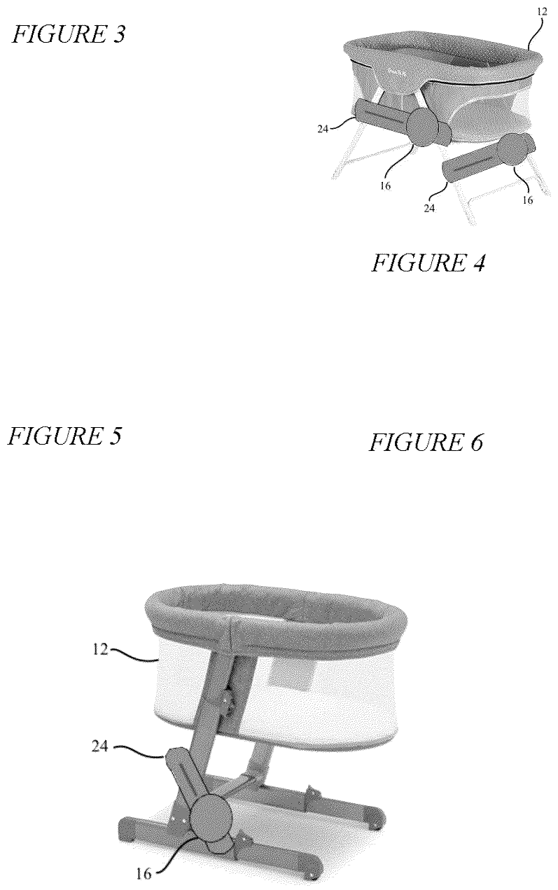

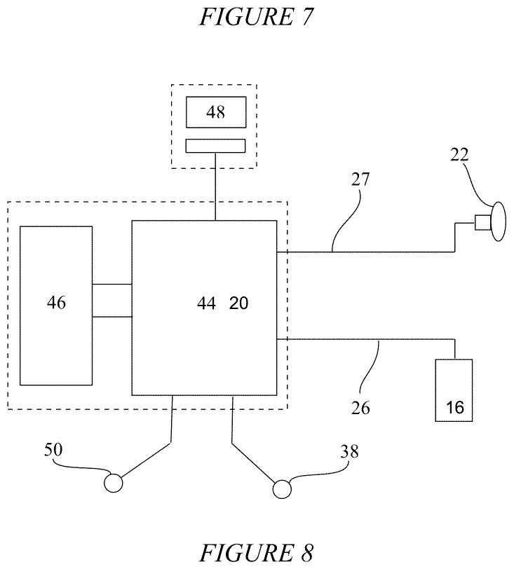

Referring to FIGS. 3-7 a variety of mounting positions for the mounting apparatus 24 on a variety of sleeping structures 12, such that the motion transducer 16 can impart vibrations to the sleeping structures 12 sufficiently to simulate the vibrations of a vehicle in motion.

Referring to FIG. 8, a diagrammatic representation of the complete baby sleep aid apparatus is shown. This includes the tactile transducer 16, the speaker 22, and the light source 38. Also shown is the vehicle simulation control unit 18, which includes an electronic control unit 44, complex audio generator 20, and a power supply 46. Also shown in this view is an SD card reader 48 for receiving pre-recorded data stream of alternative vehicle riding characteristics (different tactile motions, sounds and lights). A microphone 50 (not shown) may also be included in the vehicle simulation control unit 18, and is configured to monitor and detect a child or infant's crying sounds. Once those sounds are detected, the vehicle simulation control unit 18 may be configured to automatically activate for a predetermined duration set by the user. In the illustrated implementation, the tactile transducer 16 and speaker 22 are physically independent structures from the vehicle simulation control unit 18, while the microphone (not shown) and the light source 38 share a common housing with the vehicle simulation control unit 18. In other implementations, these features may share a common housing with the vehicle simulation control unit 18. In virtually all implementations, the tactile transducer 16 is separate from the vehicle simulation control unit 18, to avoid damage from vibration, such to allow the vehicle simulation control unit 18 to be remote from the sleeping structure 12 (FIGS. 1.3-7)

The foregoing description of the preferred embodiment of the Invention is sufficient in detail to enable one skilled in the art to make and use the invention. It is understood however, that this detail of the preferred embodiment a limit the scope of the invention, in as much as equivalents thereof and other modifications ich come within the scope of the invention as defined by the claims will become apparent to those skilled in the art upon reading this specification.

What is claimed is:

1. A sleep aid apparatus for children using a simulated vehicle motion stimulator, the apparatus comprising:
a structure having a sleeping surface sized and configured for securely confining the children;
a tactile transducer coupled to the sleep structure;
a vehicle simulation control unit is in electrical communication with the tactile transducer;
the vehicle simulation control unit is comprised of a complex audio generator configured to generate predetermined low frequency from 20-100 Hz impulse patterns and communicate the impulse patterns to the tactile transducer via a water resistant wired communication; and
a loud speaker in electrical communication with the vehicle simulating control unit, wherein the vehicle simulation control unit, when activated, simultaneously generates the predetermined low frequency from 20-100 Hz impulses that cause the tactile transducer to produce tactile motions and higher audible frequencies greater than 100 Hz that cause the speaker to produce gentle ambient internal moving motor vehicle sounds.

2. The sleep aid apparatus of claim 1 further comprising a light projector on the vehicle simulation control unit with a flexible stalk, the light projector is configured to project predetermined light patterns.

3. The sleep aid apparatus of claim 2 wherein the vehicle simulation control unit causes the light to project the predetermined light patterns simultaneously with the tactile transducer producing vibration and tactile motion in certain predetermined low frequencies from 20-100 Hz, and a loud speaker to produce the gentle ambient internal moving motor vehicles sounds in an audible range greater than 100 Hz.

4. The apparatus of claim 2 wherein the simple light projector is mounted on the vehicle simulation control unit.

5. The apparatus of claim 2, wherein the simple light projector with the flexible stalk is configured for aiming the projector in multiple directions determined by a user.

6. The apparatus of claim 2, wherein the simple light projector is configured to project a moving light pattern.

7. The apparatus of claim 1, further comprising a mounting apparatus configured for removably affixing the tactile transducer to the sleep structure.

8. The apparatus of claim 7, wherein the mounting apparatus comprises a variable distance clamp bar.

9. The apparatus of claim 1, wherein the impulse patterns generated by the tactile transducer include periodic impulse bump patterns and random steady state vibrating patterns.

10. The apparatus of claim 1, wherein the vehicle simulation control unit comprised of the complex audio amplifier is configured to have capabilities to generate and modify predetermined low-frequency tactile waves from 20-100 Hz and audible audio waves greater than 10 Hz, and accept additional pre-recorded data streams of alternate motor vehicle environment via an SD card reader.

11. The apparatus of claim 1, wherein the vehicle simulation control unit includes an internal clock, the internal clock configured to automatically shut off the apparatus after a user-defined predetermined time.

12. The apparatus of claim 1, further comprising a wireless functionality where the vehicle simulation control unit is in wireless communication with a user's mobile phone via WiFi/Bluetooth connection where the user may remotely configure and govern output signals of the vehicle control unit to modify motion characteristics and activation duration of the sleep aid apparatus.

13. The sleep aid apparatus of claim 11, wherein the vehicle simulation control unit is a device that includes wireless communication via WiFi/Bluetooth and is configured to communicate between the user's mobile phone, with an appropriate network application, and the vehicle simulation control unit.

14. The sleep aid apparatus of claim 1, wherein the sleep structure is a structure chosen from the group consisting of a baby bed, bassinet, cot, cradle, and crib.

15. The vehicle simulation control unit of claim 1 further comprising a microphone configured to detect children crying sounds with adjustable sensitivity, and automatically activate the vehicle simulation control unit for a predetermined duration set by a user.
